(19) United States
(12) Patent Application Publication
(10) Pub. No.: US 2025/0235331 A1
(43) Pub. Date: Jul. 24, 2025

(54) PROSTHETIC KNEE

(71) Applicant: College Park Industries, Inc., Warren, MI (US)

(72) Inventors: Ryan William WAHL, Warren, MI (US); Jonathan Thomas COOK, Warren, MI (US)

(21) Appl. No.: 19/027,549

(22) Filed: Jan. 17, 2025

Related U.S. Application Data
(60) Provisional application No. 63/622,335, filed on Jan. 18, 2024.

(51) Int. Cl. A61F 2/64; A61F 2/50; A61F 2/68

(52) U.S. Cl. CPC A61F 2/644; A61F 2002/5038; A61F 2002/5072; A61F 2002/6854

(57) ABSTRACT

A prosthetic knee is equipped with a lock-mode selection assembly that includes a locking member designed to restrict the rotation of the prosthetic knee at the pivot joint. The locking member is operable through both a manual lock mode selector and a remote locking mechanism. The prosthetic knee incorporates a compressible link assembly that facilitates adjustable stance flexion. The assembly features an adjustment fastener that allows for the modification of the distance over which a spring stack may be compressed.

### FIELD OF THE DISCLOSURE

The disclosure pertains to a prosthetic knee, particularly a prosthetic knee that incorporates an enhanced mode selection mechanism and an adjustable compressive linking member.

### BACKGROUND

Artificial limbs, including leg prostheses, utilize a variety of technologies to address a wide range of needs. For individuals with trans-femoral amputations, primary requirements for a leg prosthesis encompass stability during standing and throughout the stance phase of walking, mechanical compatibility with both walking and running gaits, and some degree of knee flexion during the stance and swing phases of gait.

Current prosthetic knees provide users with the ability to modify the “lock” modes of the device according to different activities. Certain designs include a manual mode selector, yet do not facilitate remote lock mode selection. Conversely, prosthetic knees that permit remote lock mode selection, such as those utilizing a lanyard and pull handle assembly, lack the capability for manual mode selection at the device itself. This limitation restricts users to a singular method of mode adjustment, as these devices do not support both options.

Another mechanism for selecting between lock modes in a prosthetic knee involves the use of a fastener that secures the device in either a locking or normal mode. However, this approach poses a disadvantage, as it does not allow for easy switching between locking and normal modes. Users who occasionally require the locking mode may prefer not to depend solely on the remote pull handle assembly to engage or disengage the device. Consequently, there exists a distinct need for a prosthetic knee equipped with a lock mode selector that can be operated both remotely and directly on the device.

Polycentric prosthetic knees typically feature a four-bar linkage with four distinct rotation axes. These devices possess an instant center of rotation (ICR) that is positioned significantly higher and posterior to the mechanical axes when the device is in an extended configuration. This design enhances stability during the stance phase and facilitates a more natural motion when sitting with a bent knee. However, polycentric prosthetic knees generally exhibit greater weight compared to single-axis knees and require more components for maintenance. Furthermore, most polycentric knees do not provide stance flexion resistance, which limits their ability to yield comfortably during sitting, on ramps, or when navigating stairs. Among those prosthetic knees that offer adjustable stance flexion, the inclusion of additional linking members, pivot points, and associated assemblies is essential to mitigate ground impact and improve the comfort of the residual limb. Unfortunately, these supplementary components often lead to increased complexity, size, and weight of the device. Thus, there is a pressing demand for a prosthetic knee device that delivers adjustable stance flexion while satisfying the ergonomic needs of the user.

### SUMMARY

A prosthetic knee is disclosed that includes a mode selector which can be operated both remotely and directly on the device. This prosthetic knee offers users the versatility to choose between remote mode selection and manual mode selection, based on their individual needs and preferences. Additionally, it features a compressible link assembly that allows for adjustable stance flexion. This design simplifies the device by reducing complexity, size, and weight, avoiding the need for extra links, pivot points, or assemblies.

In one embodiment, the prosthetic knee device consists of a housing, a chassis, a pair of anterior rods, and a posterior compressible link assembly. The anterior rods connect to the housing at upper pivot joints and to the chassis at lower pivot joints. The compressible link assembly connects to the housing at a second upper pivot joint and to the chassis at lower pivot joints. The housing contains a lock-mode selection assembly equipped with a locking member designed to halt the rotation of the compressible link assembly at the second upper pivot joint. This locking member can be operated using both a manual lock mode selector and a remote locking mechanism.

The lock-mode selection assembly features a spring that biases the locking member into a locked position against the first member of the compressible link assembly. The remote locking mechanism is activated by a pull handle on the locking member, which deforms the spring and releases the locking member from its locked position against the first member of the compressible link assembly. Therefore, while the lock is biased to remain in a locked position by the spring, the pull handle allows it to switch to an unlocked position, enabling rotation of the compressible link assembly at the second upper pivot joint.

The locking member incorporates a bracket with a slot and a pin that interfaces with a detent shaft of the lock-mode selection assembly. The detent shaft can move axially along a locking axis through the slot, which adjusts the position of the pin to alternate the locking member between locked and unlocked states. In this design, the pin is secured within the slot of the locking member and will rest against the detent shaft. When the detent shaft is moved axially, this change will either move the pin to the unlocked position or allow it to return to the locked position thanks to the spring.

Moreover, the lock-mode selection assembly ideally includes at least one indexing device (such as a spring plunger) that engages with indentations along the detent shaft. This indexing device restricts axial movement along the locking axis incrementally. The manual lock mode selector includes a button at one end of the detent shaft, allowing for manual shifting of the detent shaft along the locking axis. This button facilitates the toggling of the locking member between locked and unlocked states.

In a preferred embodiment, a button is attached to both ends of the detent shaft through a button fastener. Depending on the current mode state, one button can be pressed from one side of the prosthetic device to engage the manual lock mode selector in locking mode, while the opposite button can be pressed from the other side to switch it to unlocking mode. This assembly allows straightforward actuation of the lock on the knee, making it accessible for users who may only occasionally need to lock the knee-such as when walking in water, where the force of the water complicates knee extension. This functionality enhances the versatility of the prosthetic knee.

The compressible link assembly comprises a first member, a second member, and an inner link member positioned between them. The first member attaches to the housing at the second upper pivot joint, while the second member connects to the chassis at the second lower pivot joints. The first member contains a cylindrical bore (or chamber) through which the inner link member moves axially along a compression axis. A spring stack is situated between the first and inner link members to allow for a small degree of flexion in the prosthetic knee, particularly while standing. The second member is affixed to the inner link member and includes an adjustment fastener for modifying the compressive distance between the first member and the inner link member along the compression axis of the compressible link assembly. Thus, the compressible link assembly is adjustable.

In one embodiment, a retaining fastener secures the inner linking member inside the first member. A stop bumper is attached to the retaining fastener, while the second member connects to the inner link member. An adjustment fastener is located within the second member. When the adjustment fastener is pressed against the stop bumper, it holds the retaining fastener in place, restricting the movement of both the inner link member and the second member. In this position, the compressible link assembly does not compress the spring stack, allowing the prosthetic knee to maintain stance flexion when weight is applied.

When the adjustment fastener is moved away from the stop bumper, both the inner linking member and the lower linking member can compress the spring stack, adjusting their position relative to the upper linking member and retaining screw along the compression axis, until the stop bumper makes contact and restricts the movement of the adjustment screw.

By varying the position of the adjustment fastener, the distance that the second member and the inner link member can compress the spring stack changes, allowing for movement relative to the first member and the retaining fastener. In this configuration, the compressible link assembly is designed to compress and enable stance flexion of the prosthetic knee when weight is applied. As a result, the spring stack returns the compressible link assembly to an extended position and the prosthetic knee to its unflexed position once the weight is removed.

These numerous advantages, features, and functions of the prosthetic knee are more clearly understood through the following description and accompanying drawings.

### BRIEF DESCRIPTION OF THE DRAWINGS

These and other features, aspects, and advantages of the embodiments of the present disclosure will be better understood in the following description, appended claims, and accompanying drawings.

FIG. 1 is a rear perspective view of a prosthetic knee according to an embodiment of this disclosure.

FIG. 2 is a front-side view of the prosthetic knee, as shown in FIG. 1.

FIG. 3 is a side view of the prosthetic knee in FIG. 1.

FIG. 4 is an exploded view of the prosthetic knee in FIG. 1.

FIG. 5 is a rear perspective view of a housing and a lock-mode selection assembly according to an embodiment of this disclosure.

FIG. 6 is an exploded view of the housing in FIG. 5.

FIG. 7A-7C are perspective views of a locking member, manual lock mode selector, and remote locking mechanism of the lock-mode selection assembly according to an embodiment of this disclosure.

FIG. 8 is a rear perspective view of a compressible link assembly according to an embodiment of this disclosure.

FIG. 9 is an exploded view of the compressible link assembly, as shown in FIG. 8.

FIG. 10 is a cross-sectional view of the compressible link assembly, as shown in FIG. 8.

The drawings are not necessarily drawn to scale but instead are drawn to provide a better understanding of the components thereof and are not intended to be limiting in scope but to provide exemplary illustrations. The figures illustrate exemplary configurations of a prosthetic knee and in no way limit the structures or configurations according to the present disclosure.

### DEFINITIONS

A description of a few terms, when used, is necessary for further ease of understanding the embodiments of a prosthetic device as disclosed. As used, the term “proximal” has its ordinary meaning and refers to a location next to or near the point of attachment or origin or a central point located toward the center of the body. Likewise, the term “distal” has its ordinary meaning and refers to a location situated away from the point of attachment or origin or a central point located away from the center of the body.

Medial is toward the body's midline or the median or sagittal plane (SP), which splits the body head-to-toe into two halves, the left and right. Lateral is the side or part of the body that is away from the middle. The coronal or frontal plane (CP) divides the body into posterior (P) and anterior parts (A) and is perpendicular to the sagittal plane (SP). The term “posterior” also has its ordinary meaning and refers to a location behind or at another location's rear. The term “anterior” has its ordinary meaning and refers to a location ahead of or in front of another location.

Flexion and extension are movements that occur in the sagittal plane. They refer to increasing and decreasing the angle between two body parts: flexion is a movement that decreases the angle between two body parts. Extension refers to a movement that increases the angle between two body parts.

As used, the terms “rigid,” “flexible,” “compliant,” and “resilient” may distinguish characteristics of portions of certain features of the actuation system. The term “rigid” should denote that an element of the prosthetic device, such as a housing, generally lacks flexibility. Within the context of “rigid features,” it should indicate that they do not lose their overall shape when force is applied and may break if bent with sufficient force. The term “flexible” should denote that features are capable of repeated bending. The features may be bent into non-retained shapes or do not retain a general shape but continuously deform when force is applied. The term “resilient” may qualify as flexible features that return to an initial general shape without permanent deformation. As for the term “semi-rigid,” this term may connote properties of support members or shells that provide support and are free-standing; however, such support members or shells may have flexibility or resiliency.

The terms “approximately” or “substantially” mean that the recited characteristic, parameter, or value need not be achieved exactly but that deviations or variations, including, for example, tolerances, measurement error, measurement accuracy limitations, and other factors known to those of skill in the art, may occur in amounts that do not preclude the effect the characteristic was intended to provide. The terms “approximately” or “substantially” mean ±10% in some embodiments, ±5% in some embodiments, and ±1% in some embodiments. At the very least, and not as an attempt to limit the application of the doctrine of equivalents to the scope of the claims, each numerical parameter should be construed in light of the number of reported significant digits and by applying ordinary rounding techniques.

The term “detent shaft” refers to a quick-release pin part of the lock-mode selection assembly.

The term “fastener” refers to any suitable connecting or tightening mechanism expressly including, but not limited to, screws, bolts, and the combinations of bolts and nuts (e.g., without limitation, lock nuts) and bolts, washers and nuts.

The term “link” or “linkage” refers to a connection device, e.g., a coupling rod that unites components.

The term “pivot joint” (as in upper or lower pivot joint) refers to a structural joint having a pin, point, or short shaft on the end of which something rests and turns or upon and about which something rotates or oscillates. The disclosed pivot joints generally include pins, caps, bushings, washers, bolts, and/or O-rings.

The term “spring stack” refers to a compressive spring element loaded along its axis either statically or dynamically. A spring stack forms part of the compressible link assembly. In a preferred implementation, the spring stack is formed of distinct Belleville washers or disc springs that are conical-shaped and allows for precise customization of the load and/or deflection of the spring stack.

The term “user” refers to a person who uses the prosthetic knee. The user may be a patient or a healthcare professional.

### DETAILED DESCRIPTION OF VARIOUS EMBODIMENTS

### A. Exemplary Embodiment

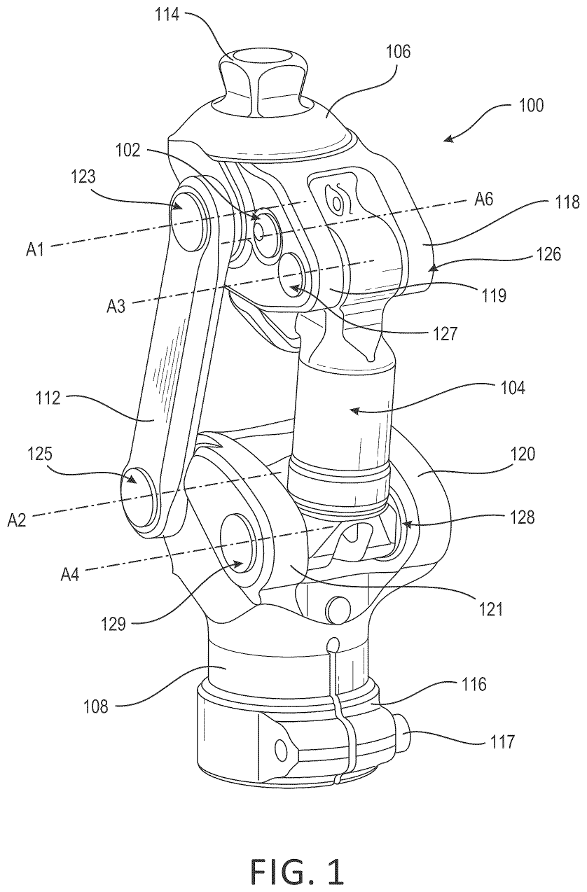

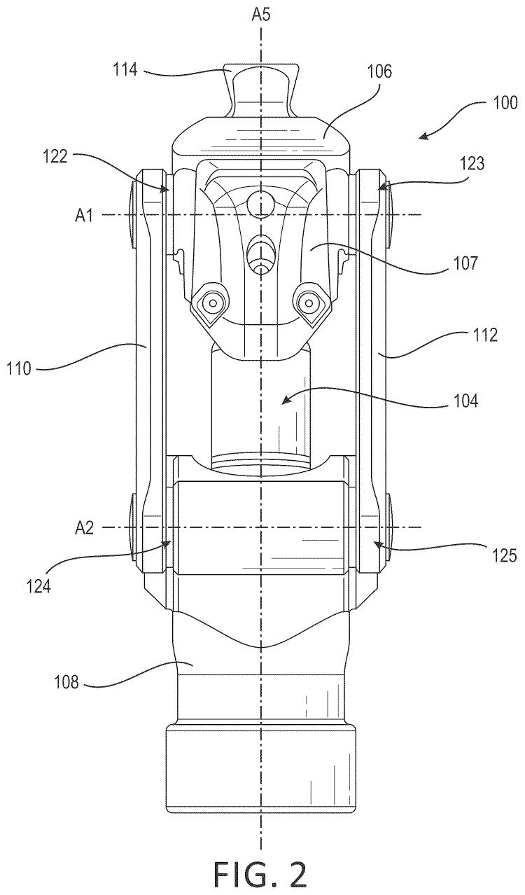

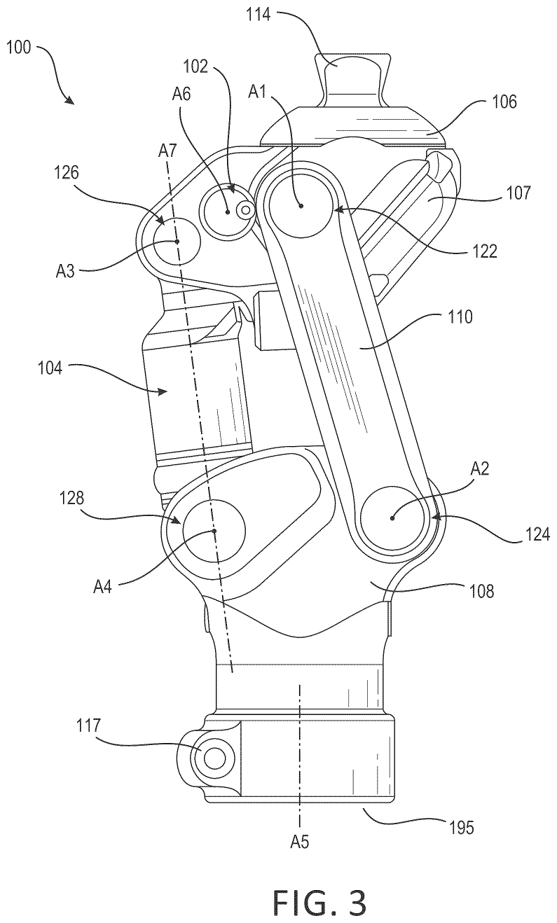

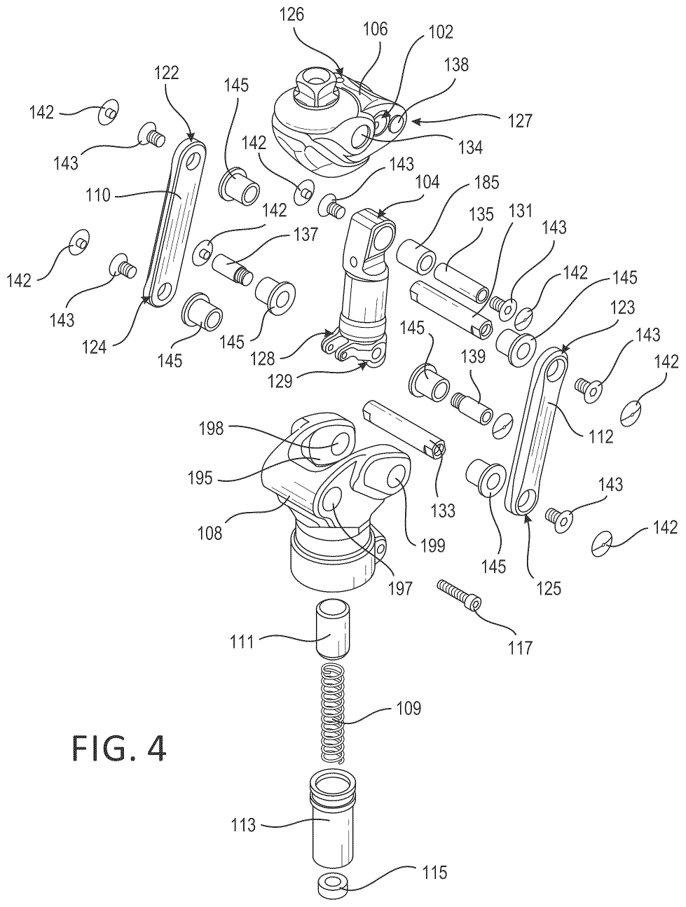

An exemplary embodiment of a prosthetic knee 100 comprising a lock-mode selection assembly 102 is shown in FIGS. 1-4. As shown, the prosthetic knee 100 has a housing 106, a chassis 108, a pair of anterior rods 110, 112, and a posterior compressible link assembly 104. In an embodiment, the prosthetic knee 100 is a polycentric knee device having a four-bar linkage construction with multiple axes of rotation. Various prosthetic knees and other features that may be combined, added, or interchanged with details described herein are described in U.S. Pat. No. 8,435,309, published on May 7, 2013, U.S. Pat. No. 9,730,814, published on Aug. 15, 2017, and U.S. Pat. No. 10,405,999, published on Sep. 10, 2019, and U.S. Patent Publication No. 2022/0280316, published on Sep. 8, 2022; the entirety of each of these documents is hereby incorporated by reference herein.

The prosthetic knee 100 includes an adapter 114 at the top housing 106. The adapter 114 may be a pyramid adapter, a 3-or 4-prong adapter, loop adapter, or Initial Knee Flexion (IKF) adapter. The housing 106 is attached to an anterior kneecap cover 107. A kneecap cover 107 is positioned along the bottom side of the housing 106 and protects the housing 106, for example, while the user is kneeling. The prosthetic knee 100 also includes a distal tube clamp attachment 116 at the bottom chassis 108. The distal tube clamp attachment 116 features a socket head cap screw (SHCS) 117 for tightening to a prosthetic leg device. The geometry of the prosthetic knee 100 advantageously allows for low voluntary control, shortening of mid-swing (which reduces stumbling risk), and geometric stability in stance.

The chassis 108 comprises a conduit 195 to contain an adjustable spring 109, the conduit 195 being aligned along a fifth axis A5 of the chassis 108. The spring 109 is housed within a tube 113 and arranged to be compressed by a plunger 111. The spring 109 is adjustable by means of an adjustment screw 115 that is rotatably secured to an inner, distal end of the tube 113. The plunger 111 is arranged to compress the spring 109 by the compressible link assembly 104. The spring force acting on the plunger 111 (and in turn the roller 190) aids in the extension of the prosthetic knee 100.

The anterior rods 110, 112 are connected to the housing 106 at first upper pivot joints 122, 123 and connected to the chassis 108 at first lower pivot joints 124, 125. The anterior rods 110, 112 are configured to rotate about a first axis A1 at the first upper pivot joints 122, 123 and configured to rotate about a second axis A2 at the first lower pivot joints 124, 125. The housing 106 also includes a friction shaft pivot hole (e.g., pivot hole 134) passing therethrough, generally parallel to the first axis A1. A first pin 131 extends through the pivot hole 134 of the housing 106 along the first axis A1 and forms part of the first upper pivot joints 122, 123. The first upper pivot joints 122, 123 each comprise ends of the first pin 131, a bearing 145, bolt 143, and cap 142.

The bolt 143 secures the anterior rod 110 to the pin 131, and the bearing 145 supports and transmits forces between the housing 106 and anterior rod 110. The cap 142 provides a protective covering for the pivot joint components. A second pin 133 extends through a hole 197 of the chassis 108 along the second axis A2 and forms part of the first lower pivot joints 124, 125. The first lower pivot joints 124, 125 each comprise ends of the second pin 133, a bearing 145, bolt 143, and cap 142. The bolt 143 secures the anterior rod 110 to the pin 133, and the bearing 145 supports and transmits forces between the chassis 108 and anterior rod 110. The first pivot joints 122, 123, 124, 125 may include one or more pins, bearings, washers, and screws to assist rotation about a respective axis A1, A2. Such pins and bearings may be of the type described in U.S. Pat. No. 9,730,814, granted Aug. 15, 2017, and incorporated herein by reference in its entirety.

The compressible link assembly 104 connects to the housing 106 at a second upper pivot joint 126 and to the chassis 108 at second lower pivot joints 128, 129. The compressible link assembly 104 is configured to rotate about a third axis A3 at the second upper pivot joint 126. The compressible link assembly 104 is likewise configured to rotate about a fourth axis A4 at the second lower pivot joints 128, 129. In an embodiment, second upper pivot joint 126 is formed by connecting the compressible link assembly 104 to the housing 106 between first and second posterior flanges 118, 119 of the housing 106. The compressible link assembly 104 is rotationally attached to the flanges 118, 119 by a third pin 135. The flanges 118, 118 protrude a main body of the housing 106 towards the posterior. The flanges 118, 118 are generally parallel and may each include a pivot pin hole (e.g., apertures 136, 138) to retain the third pin 135 and one or more bearings along the third axis A3.

The third pin 135 extends through apertures 136, 138 of the housing 106 along the third axis A3 and forms part of the second upper pivot joint 126. The second upper pivot joint 126 is formed by the connection of the flanges 118, 119 to the third pin 135, bolt(s) 143, and cap(s) 142. The bolt 143 secures the anterior rod 110 to the pin 131, and the cap 142 provides a protective covering for the pivot joint components. A bearing 185 is provided with the compressible link assembly 104 through which the third pin 135 extends. Note: the second upper pivot joint 126 may be referred to as a more than one joint 126, 127 defined by the connection of the compressible link assembly 104 and both ends of the third pin 135 at a first flange 118 and a second flange 119.

In an embodiment, the chassis 108 comprises left and right upwardly extending and generally parallel posterior ridges 120, 120. The posterior ridges 120, 120 are provided with apertures 198, 199 along the fourth axis A4 for receiving fourth and fifth pins 137, 139, respectively. The fourth and fifth pins 137, 139 are coaxial along the fourth axis A4. The second lower pivot joints 128, 129 are formed by fourth and fifth pins 137, 139, connecting the compressible link assembly 104 to the chassis 108 at the first and second posterior ridges 120, 121 of the chassis 108, respectively. The second lower pivot joints 128, 129 further comprise bearings 145 and caps. 142. In an embodiment, the pins fourth and fifth pins 137, 139 form part of the compressible link assembly 104. Alternatively, the fourth and fifth pins 137, 139 are formed as a single pin attached to the compressible link assembly 104 and chassis 108 along the fourth axis A4.

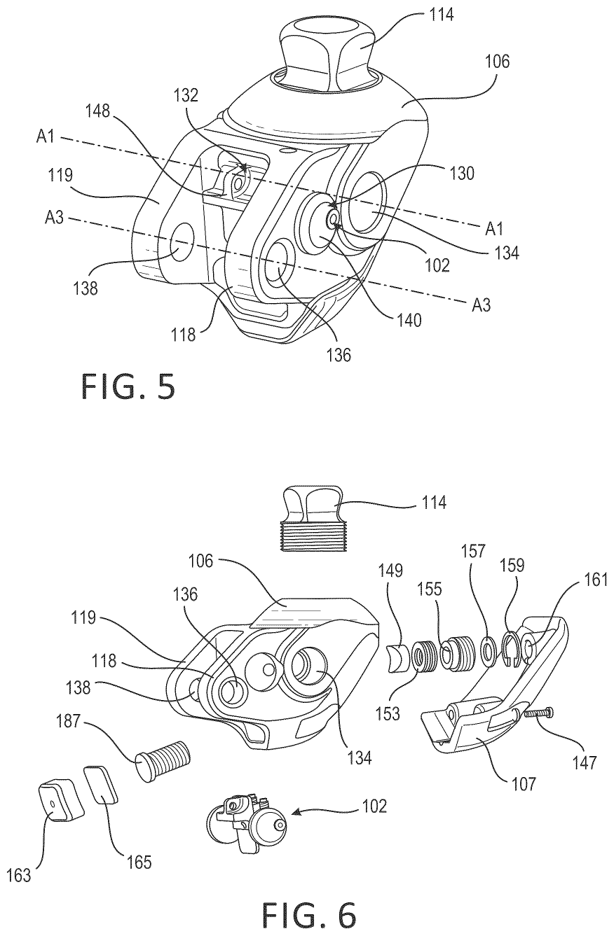

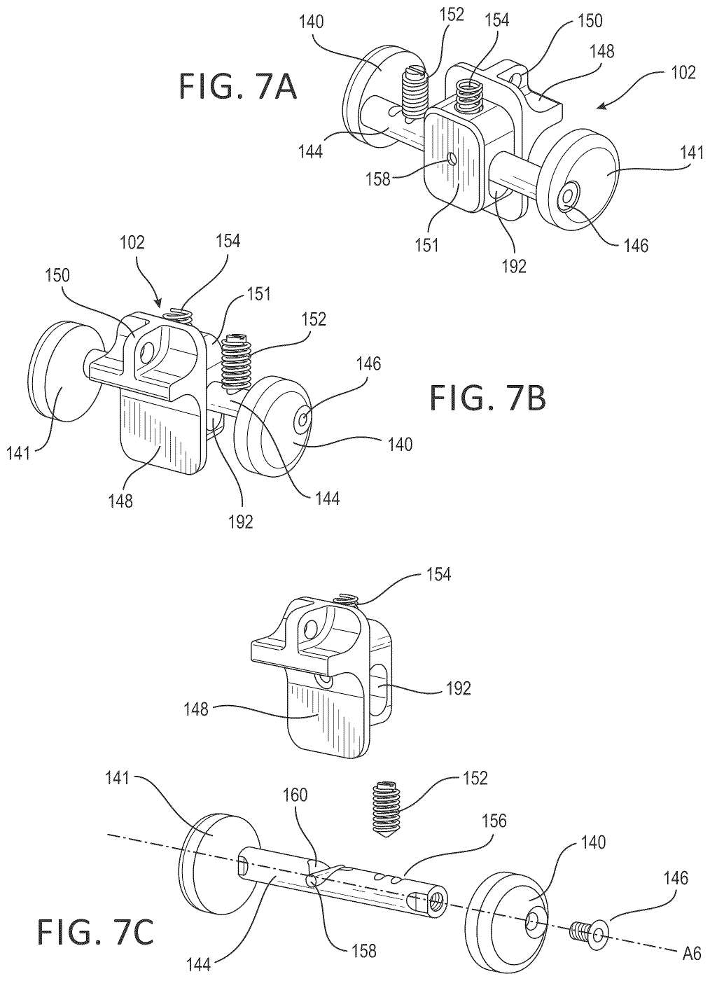

FIG. 5 illustrates a rear perspective view of the housing 106. As shown, the housing 106 includes a lock-mode selection assembly 102 with a locking member 148 that is configured to arrest rotation of the compressible link assembly 104 at the second upper pivot joint 126. The locking member 148 is operable by both a manual lock mode selector 130 and a remote locking mechanism 132. The hole 134 formed laterally through the housing 106 and along the first axis A1 is parallel to the apertures 136, 138 formed through the flanges 118, 119 and along the third axis A3. In other words, the first axis A1 and the third axis A3 are horizontal and parallel to one another. The lock-mode selection assembly 102, embedded within the housing 106, will be described in greater detail below concerning FIGS. 7A-7C.

FIG. 6 illustrates an exploded view of the housing 106. The housing 106 is attached to the kneecap cover 107 employing at least one fastener 147 (e.g., screw, bolt). Within the housing is an adjustable friction button 149, having a concave surface to rest against the first pin 131. The friction button 149 is accessible through the kneecap cover 107 via channel 161 and is adjusted using an adjustment screw 155. A spring stack 153 is provided between the friction button 149 and adjustment screw 155 to provide an adjustable level of compression and friction against the first pin 131. The spring stack 153 is preferably composed of a plurality of Belleville washers 157. A retainer ring 159 is also provided between the adjustment screw 155 and kneecap cover 107. In an alternative embodiment, the spring stack 153 is constructed as a coil spring.

The housing 106 also has a stability bumper 163 to protect the compressible link assembly 104 from impact directly against the housing 106. The stability bumper 163 may include a spacer 165 and is attached to the housing 106 by an adjustment screw 187. The adjustment screw 187 may be adjusted to vary the distance between the compressible link assembly 104 and stability bumper 163.

### B. Locking Mechanism

An exemplary embodiment of a lock-mode selection assembly 102 is shown in FIGS. 7A-7C. The lock-mode selection assembly 102 comprises a spring 154 that biases the locking member 148 in a locked position against a notch 191 of the first member 162 of the compressible link assembly 104. The remote locking mechanism 132 is actuated by a pull handle 150 formed on the locking member 148 and arranged to cause deformation of the spring 154 and release the locking member 148 from the locked position against the first member 162 of the compressible link assembly 104. The remote locking mechanism 132 may further comprise a lanyard or cable that is attached to the pull handle 150. The lanyard may be used to pull the locking member 148 in a direction opposed to the biased direction of the spring 154 for remotely unlocking the prosthetic knee 100 to enable flexion and extension of the device. The remote locking mechanism 132 thus provides a quick-release solution for unlocking rotation at the prosthetic knee 100.

The locking member 148 includes a bracket 151 and is arranged between the housing 106 and compressible link assembly 104. The locking member 148 includes a pin 158 affixed (or formed) within a slot 192 of the bracket 151 and is arranged to interface with a detent shaft 144 of the lock-mode selection assembly 102. The detent shaft 144 is arranged to axially move along a locking axis A6, within the slot 192, and adjust a relative position of the pin 158 against the detent shaft 144. The pin 158 rests against the detent shaft 144. In an embodiment, the pin 158 is perpendicular to the locking axis A6 defined by the detent shaft 144. The detent shaft 144 features at least one groove 160 corresponding to a locked and/or unlocked position, wherein the groove 160 is arranged to receive the pin 158. As such, axial movement of the detent shaft 144 will correspondingly move the pin 158 and locking member 148 into the unlocked position or allow the locking member 148 to be moved into the biased locked position by the spring 154. The detent shaft 144 may include one or more additional indentations 156 into which an indexing device 152 is engaged to retain the axial position of the detent shaft 144. In an embodiment, the indexing device 152 is a spring plunger.

In an embodiment, when the detent shaft 144 is in the unlocked position, the locking member 148 is retained in the unlocked position. Likewise, when the detent shaft 144 is in the locked position, the locking member is arranged to be biased or pushed into the locked position by the spring 154; however, the locking member 148 can also be pulled into the unlocked position by the remote locking mechanism 132.

The manual lock mode selector 130 includes at least one button 140, 141 affixed to an end of the detent shaft 144 and arranged to manually shift the detent shaft 144 along the locking axis A6. The at least one button 140, 141 is arranged to toggle the locking member 148 between locked and unlocked positions. In an exemplary embodiment, buttons 140, 141 are affixed to opposing ends of the detent shaft 144 with at least one button fastener 146. Depending on the current mode state, a first button 140 can be pushed from a first side of the prosthetic knee 100 to move the manual lock mode selector 130 into the locked mode, and, from the opposite second side of the prosthetic knee 100, a second button 141 can be pushed in the opposing direction to move the manual lock mode selector 130 into the unlocked mode.

The lock-mode selection assembly 102 strategically allows for locking and unlocking a prosthetic knee, wherein said means can be actuated directly at the device (e.g., manual lock mode selector 130) or remotely (e.g., remote locking mechanism 132). Such an assembly increases device versatility for the user. The users may thus have the option to use remote mode selection or use the mode selection on a prosthetic knee, and the users can also change how they choose to select between locking modes as their needs or abilities may change.

### C. Adjustable Stance Flexion Assembly

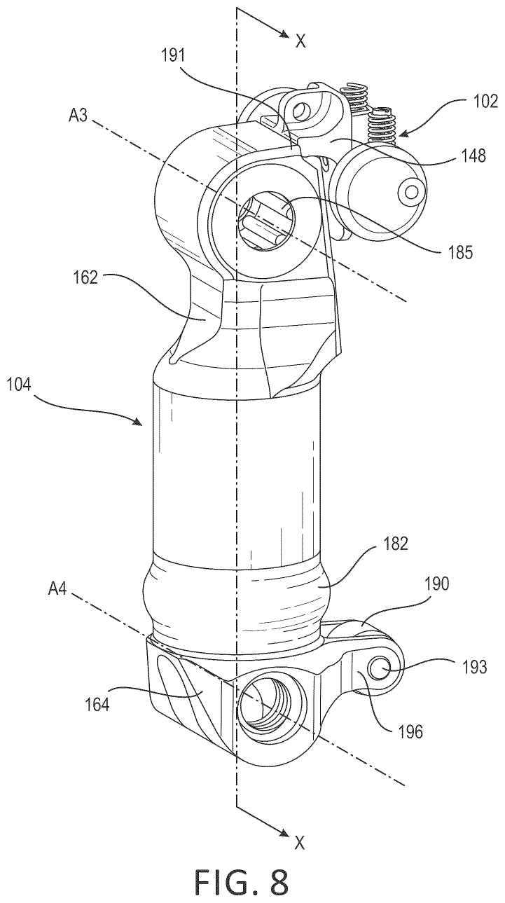

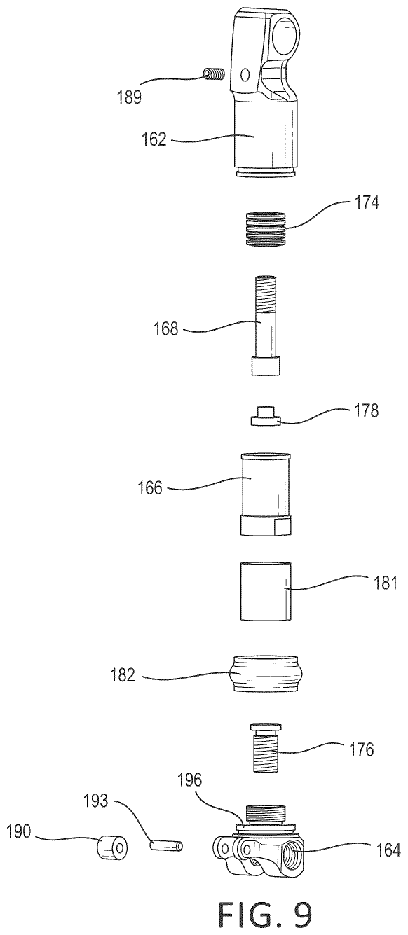

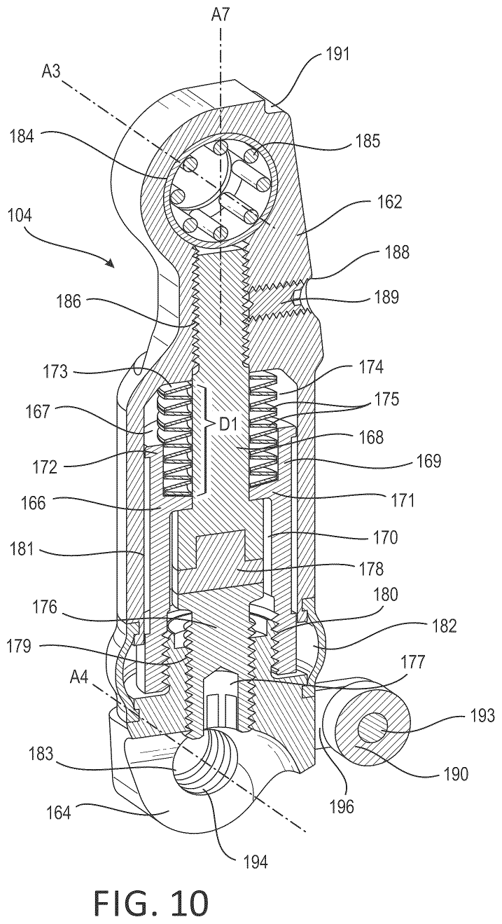

An exemplary embodiment of a lock-mode selection assembly 102 is shown in FIGS. 8-10. The disclosed prosthetic knee 100 provides a method of allowing a small amount of flexion of the prosthetic knee 100 while standing (commonly known as stance flexion) and includes means to adjust this amount of flexion. The compressible link assembly 104 comprises a notch 191 for interfacing with the locking member 148 to arrent rotation of the compressible link assembly 104 about the third axis A3. The third axis A3 is horizontal and parallel to the fourth axis A4.

As depicted, the compressible link assembly 104 includes a first member 162, a second member 164, and an inner link member 166 disposed therebetween. The first member 162 is an upper linking member attached to the housing 106 at the second upper pivot joint 126 and is disposed between the flanges 118, 119. The notch 191 is formed along an anterior surface of a first member 162 of the compressible link assembly. The formation of the notch 191 allows for translational, longitudinal movement of the locking member 148 to selectively engage and disengage the first member 162. The second member 164 is a lower linking member attached to the chassis 108 at the second lower pivot joint 128 and is disposed between the ridges 120, 121. The second member 164 comprises at least one orifice 183 to receive fourth and fifth pins 137, 139 corresponding to the second lower pivot joints 128, 129. The at least one orifice 183 includes threads 194 to receive the fourth and fifth pins 137, 139. The compressible link assembly 104 further comprises projections 196, including a 190 and pin 193 roller. The roller 190, being rotatably about the pin 193, acts on the plunger 111 that is loaded by the spring 109 to aid in the extension of the prosthetic knee 100.

The first member 162 comprises an annular opening 184 that corresponds to the third axis A3. The annular opening 184 is arranged to receive a pin that extends through and beyond apertures 136, 138 of opposing flanges 118, 119. The annular opening 184 may include a bearing 185 to facilitate rotation of the second upper pivot joint 126. The first member 162 defines a chamber 167 within which the inner link member 166 is arranged to move axially along a compression axis A7. The compression axis A7 is perpendicular to both the third axis A3 defined by the annular opening and the fourth axis A4 defined by the fourth and fifth pins 137, 139 through the orifices 183. In an embodiment, the compression axis A7 is angled from the fifth axis A5 between approximately 5 and 10 degrees.

A spring stack 174 is arranged between the first member 162 and the inner link member 166. In a preferred embodiment, the spring stack 174 comprises conical washers 175. The conical washers 175 provide improved space utilization and are cost effective. For example, in considering fatigue life of the spring elements, the price to replace a conical washer within the spring stack is more cost effective than the price to replace an entire coil spring. In an alternative embodiment, the spring stack 174 comprises an elastomer or another type of spring element (e.g., coil spring).

In an embodiment, the inner link member 166 includes a first receptable to hold the spring stack 174 within the chamber 167. A retainer member 168, configured to extend through the spring stack 174 along the compression axis A7, constrains the inner link member 166 within the chamber 167 of the first member 162. In an embodiment, the retainer member 168 is threaded into a first channel 186 of the first member 162, the first channel 186 being superior to the chamber 167 and inner link member 166. A second channel 188 may be formed in the first member 162, being perpendicular to the first channel 186, and arranged to receive a lateral fastener 189. The lateral fastener 189 is arranged to interlock with threads of the retainer member 168 and may be used to adjust a longitudinal position of the retainer member 168 within the chamber 167.

A stop bumper 178 is affixed to the retainer member 168 within a second receptacle 170 of the inner link member 166. The stop bumper 178 may be composed of an elastic, flexible material such as rubber. An inner flange 171 of the inner link member 162 divides the first receptacle 169 from the second receptacle, wherein the retainer member 168 is arranged to extend through an opening of the inner flange 171 along the compression axis A7. In an embodiment, a bushing 181 is arranged within the chamber 167 and between the inner link member 166 and the first member 162. The bushing 181 may be restricted in the axial direction by an outer flange 172 of the inner link member 166.

The second member 164 is affixed to the inner link member 166. In an embodiment, a threaded outer surface 180 of the second member 164 is configured and dimensioned to interface with the second receptacle 170 of the inner link member 166. An adjustment fastener 176 is arranged within the second member 164 and configured to adjust a compressive distance D1 between an upper chamber surface 173 of the first member 162 and the inner flange 171 of the inner link member 166 along the compression axis A7 of the compressible link assembly 104.

The adjustment fastener 176 comprises a screw drive 177 (i.e., groove) configured and dimensioned to receive an external tool (e.g., screwdriver) to tighten and loosen the adjustment fastener 176 against an inner surface 179 of the second member 164 and along the compression axis A7. In an embodiment, the compressible link assembly 104 includes an outer seal 182 arranged to protect an exposed portion of the inner link member 166 between the first member 162 and the second member 164. The outer seal 182 may be composed of a flexible, elastic material.

When the adjustment fastener 176 is seated against the stop bumper 178, the retainer member 168 is held in place to constrain the movement of the inner link member 166 and second member 164. In this state, the compressible link assembly 104 will not compress and provide stance flexion of the prosthetic knee 100 when weight is applied.

When the adjustment fastener 176 is moved away from the stop bumper 178, the inner link member 166 and the second member 164 can compress the spring stack 174 and move relative to the first member 162 and retainer member 168 a distance up along the compression axis A7 until the stop bumper 178 abuts the adjustment fastener 176. Thus, varying the axial position of the adjustment fastener 176 changes the amount of distance that the second member 164 and inner link member 166 can compress the spring stack 174 and move relative to the first member 162 and retainer member 168. In this state the compressible link assembly 104 can compress and provide stance flexion of the prosthetic knee 100 when weight is applied. The spring stack 174 returns the compressible link assembly 104 to its extended position and the prosthetic knee 100 to its unflexed position when weight is removed.

In an alternative embodiment, the compressible link assembly 104 is configured to use external springs (i.e., springs outside of the chamber 167). Additionally, the compressible link assembly 104 may be configured to use an external adjustment fastener 176 to adjust a distance (D1) that a spring stack 174 (e.g., internal or external) may be compressed.

It will be recognized that the prosthetic knee and components thereof can be made from any suitable materials. For example, the components can be constructed from an appropriate material such as those capable of providing lightweight structural support. Examples of such materials include, but are not limited to, plastics, steel alloys, aluminum alloys, other metals, ceramics, or other rigid materials. In an exemplary embodiment, the chassis, housing, anterior rods, and posterior link may be made from machined aluminum (2024) and may be anodized, for example, black or grey. Further, in the exemplary embodiment, the extension stop bumper can be made of rubber and various springs can be made from spring steel.

Of course, it is to be understood that not necessarily all objects or advantages may be achieved under any embodiment of the disclosure. Those skilled in the art will recognize that the disclosed prosthetic knee may be embodied or carried out to achieve or optimizes one advantage or group of advantages as taught herein without achieving other objects or advantages as taught or suggested herein. The disclosed prosthetic knee 100 is generally described concerning a polycentric design, though the teachings of the lock-mode selection assembly and compressible link assembly can apply to other prosthetic knee devices that are not polycentric.

The skilled artisan will recognize the interchangeability of various disclosed features. Besides the variations described herein, other known equivalents for each feature can be mixed and matched by one of ordinary skill in this art to build and use prosthetic devices under principles of the present disclosure. It will be understood by the skilled artisan that the features described herein may be adapted to other methods and types of prosthetic devices or applications.

It is intended that the present disclosure should not be limited by the disclosed embodiments described above and may be extended to other applications that may employ the features described herein.

1. A prosthetic knee comprising:
a housing;
a chassis;
a pair of anterior rods connecting the housing to the chassis, the anterior rods connected to the housing at first upper pivot joints and to the chassis at first lower pivot joints; and
a posterior compressible link assembly connecting the housing to the chassis, the compressible link assembly connected to the housing at a second upper pivot joint and to the chassis at second lower pivot joints;
wherein the housing includes a lock-mode selection assembly having a locking member arranged to arrest rotation of the compressible link assembly at the second upper pivot joint;
wherein the locking member is operable by both a manual lock mode selector and a remote locking mechanism.

2. The prosthetic knee of claim 1, wherein the lock-mode selection assembly comprises a spring arranged to bias the locking member in a locked position against a first member of the compressible link assembly.

3. The prosthetic knee of claim 2, wherein the remote locking mechanism is actuated by a pull handle formed on the locking member and arranged to cause deformation of the spring and release the locking member from the locked position against the first member of the compressible link assembly.

4. The prosthetic knee of claim 2, wherein the locking member includes a bracket and a pin arranged to interface with a detent shaft of the lock-mode selection assembly, the detent shaft arranged to axially move along a locking axis and adjust a relative position of the pin to switch the locking member between locked and unlocked positions.

5. The prosthetic knee of claim 4, wherein the lock-mode selection assembly further comprises at least one indexing device to engage with at least one indentation of the detent shaft to restrict axial movement along the locking axis.

6. The prosthetic knee of claim 5, wherein the manual lock mode selector includes at least one button affixed to an end of the detent shaft and arranged to manually shift the detent shaft along the locking axis and toggle the locking member between locked and unlocked positions.

7. The prosthetic knee of claim 1, wherein the compressible link assembly includes a first member, a second member, and an inner link member disposed between the first member and the second member;
wherein the first member is attached to the housing at the second upper pivot joint and the second member is attached to the chassis at the second lower pivot joints; and
wherein the first member defines a chamber within which the inner link member is arranged to move axially along a compression axis.

8. The prosthetic knee of claim 7, wherein a spring stack is arranged between the first member and the inner link member.

9. The prosthetic knee of claim 8, wherein the compressible link assembly is arranged to provide stance flexion of the prosthetic knee by compression of the spring stack along the compression axis and between the first member and the inner link member.

10. The prosthetic knee of claim 7, wherein the second member is affixed to the inner link member and includes an adjustment fastener arranged to adjust a distance between the first member and the inner link member along the compression axis of the compressible link assembly.

11. A prosthetic knee comprising:
a housing;
a chassis;
a pair of anterior rods connecting the housing to the chassis, the anterior rods connected to the housing at first upper pivot joints and to the chassis at first lower pivot joints; and
a posterior compressible link assembly connecting the housing to the chassis, the compressible link assembly connected to the housing at a second upper pivot joint and to the chassis at second lower pivot joints;
wherein the compressible link assembly includes a first member, a second member, and an inner link member disposed between the first member and the second member;
wherein the first member is attached to the housing at the second upper pivot joint and the second member is attached to the chassis at the second lower pivot joints;
wherein the first member defines a chamber within which the inner link member is arranged to move axially along a compression axis.

12. The prosthetic knee of claim 11, wherein a spring stack is arranged between the first member and the inner link member.

13. The prosthetic knee of claim 12, wherein the compressible link assembly is arranged to provide stance flexion of the prosthetic knee by compression of the spring stack along the compression axis and between the first member and the inner link member.

14. The prosthetic knee of claim 11, wherein the second member is affixed to the inner link member and includes an adjustment fastener arranged to adjust a distance between the first member and the inner link member along the compression axis of the compressible link assembly.

15. The prosthetic knee of claim 11, wherein the housing includes a lock-mode selection assembly having a locking member arranged to arrest rotation of the compressible link assembly at the second upper pivot joint, the locking member being operable by both a manual lock mode selector and a remote locking mechanism.

16. The prosthetic knee of claim 15, wherein the lock-mode selection assembly comprises a spring to bias the locking member in a locked position against a first member of the compressible link assembly.

17. The prosthetic knee of claim 16, wherein the remote locking mechanism is actuated by a pull handle formed on the locking member and arranged to cause deformation of the spring and release the locking member from the locked position against the first member of the compressible link assembly.

18. The prosthetic knee of claim 16, wherein the locking member includes a bracket and a pin arranged to interface with a detent shaft of the lock-mode selection assembly, the detent shaft arranged to axially move along a locking axis and adjust a relative position of the pin to switch the locking member between locked and unlocked positions.

19. The prosthetic knee of claim 18, wherein the manual lock mode selector includes at least one button affixed to an end of the detent shaft and arranged to manually shift the detent shaft along the locking axis and toggle the locking member between locked and unlocked positions.

20. A prosthetic knee comprising:
a housing;
a chassis;
a pair of anterior rods connecting the housing to the chassis, the anterior rods connected to the housing at first upper pivot joints and to the chassis at first lower pivot joints; and
a posterior compressible link assembly connecting the housing to the chassis, the compressible link assembly connected to the housing at a second upper pivot joint and to the chassis at second lower pivot joint;
wherein the housing includes a lock-mode selection assembly having a locking member arranged to arrest rotation of the compressible link assembly at the second upper pivot joint;
wherein the locking member is operable by both a manual lock mode selector and a remote locking mechanism;
wherein the compressible link assembly includes a first member, a second member, and an inner link member disposed between the first member and the second member;
wherein the first member is attached to the housing at the second upper pivot joint and the second member is attached to the chassis at the second lower pivot joints;
wherein the first member defines a chamber within which the inner link member is arranged to move axially along a compression axis.
